(19) United States
(12) Patent Application Publication
(10) Pub. No.: US 2017/0239434 A1
(43) Pub. Date: Aug. 24, 2017

(54) TRACHEAL TUBES AND SEALS

(71) Applicant: SMITHS MEDICAL INTERNATIONAL LIMITED, Kent (GB)

(72) Inventors: Timothy Bateman, Kent (GB); Stephen James Field, Kent (GB); Andrew Thomas Jeffrey, Kent (GB)

(73) Assignee: SMITHS MEDICAL INTERNATIONAL LIMITED, Kent (GB)

(21) Appl. No.: 15/511,659

(22) PCT Filed: Aug. 21, 2015
(86) PCT No.: PCT/GB2015/000252

§ 371 Date: Mar. 16, 2017

(30) Foreign Application Priority Data
Sep. 25, 2014 (GB) 1416891.8

(51) Int. Cl. A61M 16/04

(52) U.S. Cl. CPC A61M 16/0436; A61M 16/0459; A61M 16/044; A61M 2205/02

(57) ABSTRACT

A tracheostomy tube has an inflatable sealing cuff (11) towards its patient end (6) and a fluid-permeable cuff (31) mounted on the shaft (1) above the inflatable cuff. The fluid-permeable cuff (31) contains hydrogel granules (32) that absorb moisture in the trachea (T) and help reduces the amount of secretions leaking past the sealing cuff (11). When the tube is to be withdrawn from the trachea (T) the lower, inflatable cuff (11) is deflated allowing the upper, fluid-permeable cuff (31) to be flattened against the outside of the lower cuff.

This invention relates to tracheal tubes of the kind having a shaft and an expansible sealing member mounted on the shaft towards its patient end.

Tracheal tubes are used to supply ventilation and anaesthetic gases to a patient, such as during surgery. The tracheal tube may be inserted via the mouth or nose, in the case of an endotracheal tube, or may be inserted via a surgically-made tracheostomy opening in the neck, in the case of a tracheostomy tube. Most, but not all, tracheal tubes have some form of a seal on their outside which forms a seal between the outside of the tube and the inside of the trachea so that gas flow is confined to the bore of the tube and cannot flow around the outside of the tube, between the tube and the trachea.

The most common form of seal is provided by an inflatable cuff that is inflated and deflated via a small bore lumen extending along the tube and connected towards its rear end to an inflation line terminated by an inflation indicator, valve and connector. These inflatable cuffs may be of the high-volume/low-pressure kind where the cuff is formed of a flexible plastics material moulded with a natural cylindrical or doughnut shape that is inflated without stretching, to contact the wall of the trachea, by relatively low-pressure gas supplied via the inflation line. Alternatively, the cuff may be of the low- volume/high-pressure kind where the cuff is of an elastic material that lies close to the tube shaft when uninflated but is inflated and stretched to a larger diameter by relatively high pressure gas supplied via the inflation line. These cuffs can also be inflated with a liquid. Various problems exist with both forms of cuff. One problem is the difficulty of preventing secretions that collect above the cuff leaking between the cuff and the trachea and entering the bronchial passages. The leakage of such secretions is thought to contribute to ventilator-associated pneumonia (VAP).

Various suggestions have been made for cuffs intended to reduce leakage. US2009/0092902 describes a cuff for an endotracheal tube that is coated with a hydrophobic or dirt-repellent compound. WO2008/109009 describes an endotracheal tube having a cuff having a water-swellable outer layer that acts to limit leakage past the cuff. U.S. Pat. No. 7,950,393 describes an endotracheal tube with a cuff having filtration layer on its outer surface intended to filter microbes. U.S. Pat. No. 6,526,977 and U.S. Pat. No. 6,802,317 describe thin sealing cuffs of polyurethane. WO99/44665 describes a seal made entirely of a hydrogel material. WO2008/072102 describes an arrangement where a liquid gel is poured on top of the sealing cuff so that it sets in position.

It is an object of the present invention to provide an alternative tracheal tube.

According to the present invention there is provided a tracheal tube of the above-specified kind, characterised in that the tube also includes a fluid-absorbing component mounted on the shaft on the machine side of the sealing member, and that the fluid-absorbing component is provided by a fluid-absorbing substance within an outer fluid- permeable cuff.

The fluid-absorbing substance may include a hydrogel. The fluid-absorbing substance is preferably in particulate form, such as in granules or powder. The fluid-absorbing component is preferably arranged to expand radially initially on exposure to moisture until it contacts the wall of the trachea and subsequently to expand along the shaft away from the patient end. The radial width of the fluid-absorbing component is preferably greater than its axial length. The expansible sealing member may be an inflatable cuff. The lower side of the fluid-absorbing component is preferably in contact with the upper end of the expansible sealing member. The fluid-absorbing component is preferably arranged to be folded against the outside of the expansible sealing member when the expansible sealing member is deflated for withdrawal of the tube from the trachea after use.

The tracheal tube may be a tracheostomy or endotracheal tube.

A tracheostomy tube according to the present invention will now be described, by way of example, with reference to the accompanying drawings, in which:

FIG. 1 is a side elevation view of the tube;

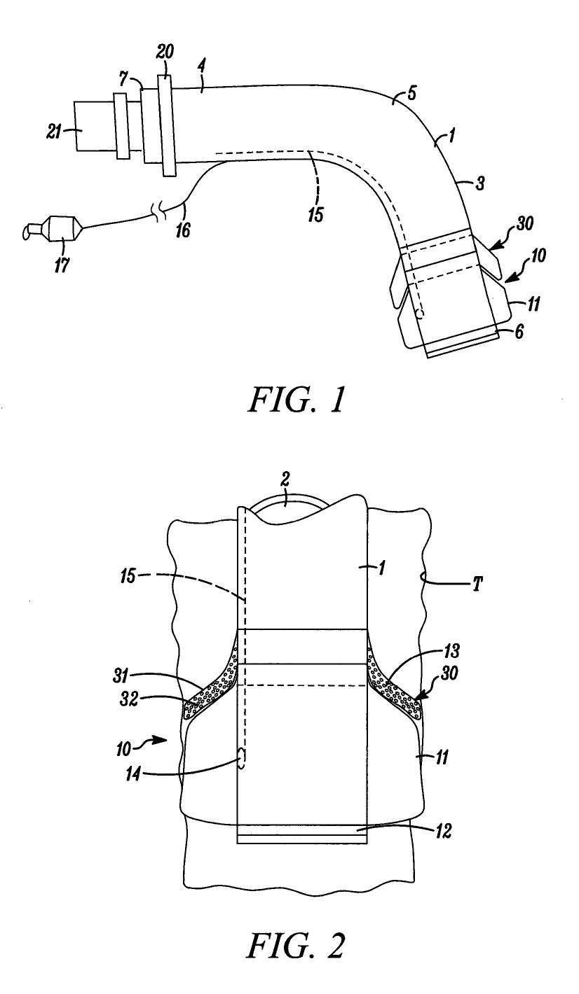

FIG. 2 is an enlarged side elevation of the tube in a trachea in the region of the sealing cuff;

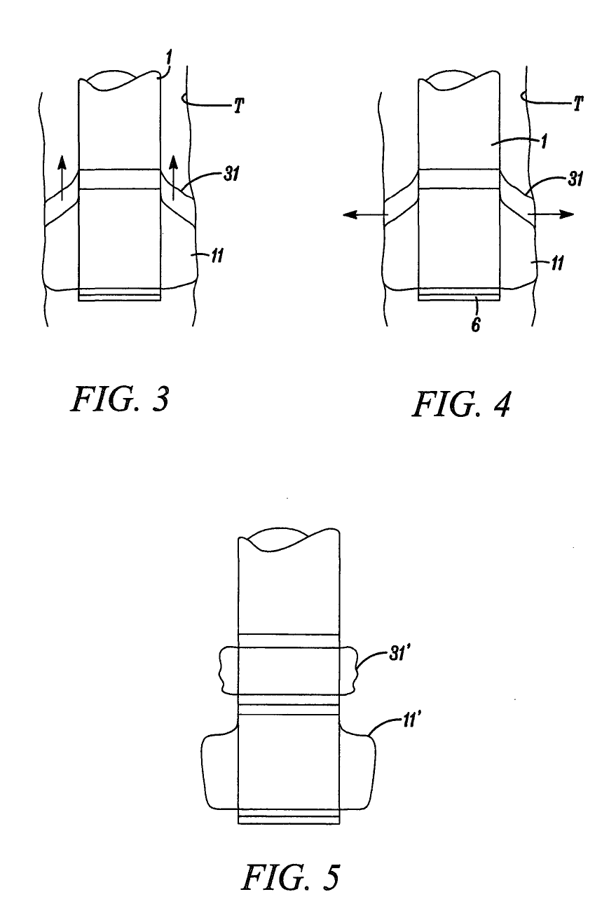

FIGS. 3 and 4 show the sealing cuff at successive stages of use; and

FIG. 5 is a side elevation view of a modified form of tube.

With reference first to FIGS. 1 and 2, the tracheostomy tube includes a tubular shaft 1 having a bore 2 extending along its length. The tube is formed with a relatively straight patient end portion 3 and a relatively straight machine end portion 4 linked by a curved intermediate portion 5 so that the patient and machine ends 6 and 7 are angled at about 100° to one another. The shaft 1 is extruded or moulded from a plastics material such as PVC. At its machine end 7 the tube has a flange 20, to which a neck strap (not shown) is attached, and a standard 15 mm female coupling 21.

Towards its patient end 6 the tube has sealing means indicated generally by the numeral 10. The sealing means 10 has two components, the first of which is provided by an expansible sealing member in the form of an inflatable first cuff 11 embracing the shaft 1. The cuff 11 is of the high-volume/low-pressure kind so that it has a relatively floppy shape when deflated but, when inflated with air, it fills out at low pressure to a diameter matching the internal diameter of the trachea T. The cuff 11 may be of PVC or any other conventional material. The cuff 11 is attached to the shaft 1 by two collar portions 12 and 13 at opposite ends. The cuff 11 extends over an opening 14 on the outer surface of the shaft 1 into an inflation lumen 15 extending along the shaft within its wall thickness. The inflation lumen 15 is connected towards the rear end of the tube with a small-bore inflation line 16 that is terminated by a combined inflation indicator or pilot balloon, valve and connector 17. The cuff 11 is inflated and deflated with air by connecting an air-filled syringe (not shown) with the connector 17. Alternatively, the cuff 11 could be inflated with a liquid, such as saline. Other types of expansible sealing member could be used, such as, for example, a foam cuff that expands by the resilience of the foam and is sucked down to a smaller diameter for insertion and removal by using a syringe or the like.

The other component of the sealing means 10 is provided by separate fluid-absorbing means or component 30 mounted on the shaft 1 just above the cuff 11, that is, on the machine side of the cuff and lies, in use, in contact with the upper, machine side of the cuff. The fluid-absorbing means 30 consists of a flexible bag or second cuff 31 of a fluid-permeable material (or rendered fluid-permeable by perforations) and containing a fluid-absorbing substance 32 such as a swellable hydrogel in granule or powder form. Other fluid-absorbing substances could be used. The size and shape of the filled cuff 31 is such that, before use, it can be easily inserted into the trachea when the inflatable cuff 11 is in a deflated state. This can be ensured by choice of a suitable volume of the hydrogel 32 and by providing this in a particulate state so that the cuff 31 and its contents can be readily deformed for insertion.

Initially, when the tube has been inserted in the trachea T and the first cuff 11 is inflated, the second cuff 31 remains a relatively loose fit within the trachea and so provides little or no sealing effect. During use, however, the second cuff 31 is exposed to moisture in secretions that collect in the trachea T above the sealing means 10. Some of this moisture is absorbed through the wall of the cuff 31 into the hydrogel material 32 inside the cuff, thereby causing the hydrogel to expand. Initially, the hydrogel 32 in its cuff 31 expands radially outwardly until it contacts and applies a light pressure to the wall of the trachea T, as illustrated in FIG. 3. When expanded, the radial width of the cuff 31, that is the radial dimension between the outer surface of the shaft 1 and the perimeter of the cuff, is greater than its axial length, that is, the length of the cuff along the axis of the shaft, so that it is of annular rather than cylindrical form. As the cuff 31 and its contents 32 are exposed to further moisture, the hydrogel and its cuff expand slightly rearwardly along the shaft 1, away from the patient end 6 so that substantially no additional pressure is applied to the trachea, as illustrated in FIG. 4. The lower, patient end of the cuff 31 rests on the upper, machine end of the lower cuff 11 as the upper cuff expands. The expanded, hydrogel-filled cuff 31 effectively reduces passage of secretions past the lower inflated cuff 11. This is achieved by two mechanisms. First, the light pressure exerted by the expanded cuff 31 on the wall of the trachea T forms an additional mechanical barrier above the lower, inflatable cuff 11. Second, the amount of liquid secretions is reduced because a large part of these, or the moisture, they contain is absorbed into the hydrogel material 32 so there is a smaller volume of secretions to leak past the sealing means 10.

In order to remove the tracheal tube, the lower cuff 11 is first deflated. This then enables the outer edge of the gel-filled cuff 31 to be folded down, deformed or flattened against the outside of the deflated cuff 11 as the tube is pulled outwardly. In this way, the gel-filled cuff 31 and its contents do not form any significant protrusion on the shaft 1 to hinder withdrawal. It will be appreciated that, if a gel-filled cuff were used to provide the sole seal, it would have to be long enough to form an effective gas seal. This could make it very difficult to remove the tube after use. By combining a cuff or bag filled with a fluid-absorbing material with a conventional expansible sealing cuff it is possible to facilitate removal whilst also achieving an enhanced seal.

Instead of mounting the fluid-absorbing cuff directly in contact with the inflatable cuff, as in the arrangement described above, a fluid-absorbing cuff 31′ could be spaced a short distance rearwardly of the expansible cuff 11′, further towards the machine end, as shown in FIG. 5, although this might present more difficulties during removal.

1-8. (canceled)

9. A tracheal tube having a shaft and an expansible sealing member mounted on the shaft towards its patient end, characterised in that the tube also includes a fluid-absorbing component mounted on the shaft on the machine side of the sealing member, and that the fluid-absorbing component is provided by a fluid-absorbing substance within an outer fluid-permeable cuff.

10. A tracheal tube according to claim 9, characterised in that the fluid-absorbing substance includes a hydrogel.

11. A tracheal tube according to claim 9, characterised in that the fluid-absorbing substance is in particulate form, such as in granules or powder.

12. A tracheal tube according to claim 9, characterised in that the fluid-absorbing component is arranged to expand radially initially on exposure to moisture until it contacts the wall of the trachea and subsequently to expand along the shaft away from the patient end.

13. A tracheal tube according to claim 9, characterised in that the radial width of the fluid-absorbing component is greater than its axial length.

14. A tracheal tube according to claim 9, characterised in that the expansible sealing member is an inflatable cuff.

15. A tracheal tube according to claim 9, characterised in that the lower side of the fluid-absorbing component is in contact with the upper end of the expansible sealing member.

16. A tracheal tube according to claim 9, characterised in that the fluid-absorbing component is arranged to be folded against the outside of the expansible sealing member when the expansible sealing member is deflated for withdrawal of the tube from the trachea after use.
